(19) United States
(12) Patent Application Publication
(10) Pub. No.: US 2007/0197648 A1
(43) Pub. Date: Aug. 23, 2007

(54) Pharmaceutical product containing tranilast

(75) Inventors: Motoyoshi Inooka, Osaka-shi (JP); Tadashi Seto, Osaka-shi (JP)

Correspondence Address: FITCH, EVEN, TABIN & FLANNERY, P. O. BOX 18415, WASHINGTON, DC, 20036, US

(73) Assignee: Rohto Pharmaceutical Co., Ltd., Osaka (JP)

(21) Appl. No.: 10/594,760

(22) PCT Filed: Mar. 29, 2005
(86) PCT No.: PCT/JP05/05967

§ 371 Date: Sep. 29, 2006

(30) Foreign Application Priority Data
Mar. 30, 2004 (JP) 2004-098542
Mar. 31, 2004 (JP) 2004-107022
Mar. 31, 2004 (JP) 2004-107023
Apr. 15, 2004 (JP) 2004-120771
Apr. 15, 2004 (JP) 2004-120772

(51) Int. Cl. A61K 31/196

(52) U.S. Cl. 514/563; 206/570

(57) ABSTRACT

The invention provides a pharmaceutical product in which a tranilast-containing pharmaceutical preparation is contained in a packaging container through which the content can be visually observed and which can inhibit photodegradation of tranilast. The invention provides a pharmaceutical product in which a pharmaceutical preparation containing tranilast and/or a salt thereof is contained in a packaging container through which the content can be visually observed and which is provided with a shading means for blocking light in the wavelength range from 350 nm to 450 nm.

### TECHNICAL FIELD

The invention relates to tranilast-containing pharmaceutical products. More specifically, the invention relates to pharmaceutical products in which a tranilast-containing pharmaceutical preparation is contained in a packaging container through which the content can be visually observed and which can prevent the photodegradation of tranilast. The invention relates to methods for inhibiting the photodegradation of tranilast.

### BACKGROUND OF THE INVENTION

Tranilast (N-(3,4-dimethoxycinnamoyl)anthranilic acid is orally administered for treating allergic conditions, such as bronchial asthma, allergic rhinitis, etc. Tranilast is also presently used as eye drops for treating allergic diseases. In general, since tranilast is extremely unstable when exposed to light, it is very important to ensure the stability of a tranilast-containing pharmaceutical preparation when exposed to light during manufacture and/or after it is opened. The stability of preparations containing pharmaceutically active substances that are unstable to light is usually ensured by pouring such preparations into brown or aluminum containers and/or by keeping the container holding such preparations in a bag to prevent light penetration.

It is preferable to hold pharmaceutical preparations, such as eye drops, eyewashes, injections, etc., in a transparent container through which the content can be visually observed to make it possible to check the presence of foreign substances. Moreover, it is also desirable for users that containers for containing pharmaceutical preparations are transparent to some degree so the content can be observed with the naked eye to check the remaining amount.

Heretofore, brown glass containers have been used for holding tranilast-containing pharmaceutical preparations. However, such existing brown containers posed a problem in that the stability of tranilast against light cannot be sufficiently ensured. Also, aluminum or opaque containers capable of substantially blocking light have a disadvantage in that the state or amount of pharmaceutical preparations stored therein cannot be checked from the outside of the container, which causes inconvenience in manufacturing process management and quality control. Moreover, it is extremely inconvenient for users to carry/keep tranilast-containing pharmaceutical preparations in containers placed inside bags which do not allow light to pass through, although the stability of tranilast can be ensured.

Patent document 1, for example, proposes a method for improving the photostability of drugs from the viewpoint of the formulation of pharmaceutical preparations; however, it does not disclose any means for maintaining the stability of drugs against light by modifying a packaging container for storing drugs.

In view of the prior art, the development of tranilast-containing pharmaceutical preparations held in a container through which the content can be visually observed and which can prevent the photodegradation of tranilast has been desired.

Patent Document 1: Japanese Unexamined Patent Publication No. 2003-26575

### DISCLOSURE OF THE INVENTION

### Problem to be Solved by the Invention

The invention aims to provide a pharmaceutical product for holding a tranilast-containing pharmaceutical preparation in a container through which the content can be visually observed and which can inhibit the photodegradation of tranilast. The invention also aims to provide a method for inhibiting the photodegradation of tranilast.

### Means for Solving the Problem

The inventors carried out extensive research in view of the above problems and found that the degradation of tranilast when exposed to light is caused by light in the wavelength range from 365 nm to 430 nm and thus the photostabilization of tranilast is achieved by blocking light within this range. The invention was accomplished by further studies based on these findings.

More specifically, the invention provides the following pharmaceutical products.

Item 1. A pharmaceutical product, comprising a pharmaceutical preparation containing tranilast and/or a salt thereof in a packaging container through which the contents are visible and which blocks light in the wavelength range from 365 nm to 430 nm.

Item 2. A pharmaceutical product according to Item 1, wherein the packaging container has an average light transmittance of 20% or lower in the wavelength range from 365 nm to 430 nm.

Item 3. A pharmaceutical product according to Item 2, wherein the packaging container has an average light transmittance of 20% or lower in the wavelength range from 350 nm to 430 nm.

Item 4. A pharmaceutical product according to Item 1, wherein the packaging container has an average light transmittance of 20% or lower in the wavelength range from 350 nm to 450 nm.

Item 5. A pharmaceutical product according to any one of Items 1 to 4, wherein the packaging container has an average light transmittance of 20% or lower in the wavelength range from 365 nm to 430 nm, 20% or lower in the wavelength range from 350 nm to 430 nm, and 20% or lower in the wavelength range from 350 nm to 430 nm.

Item 6. A pharmaceutical product according to any one of Items 1 to 5, wherein a part of the packaging container has an average light transmittance of 30% or higher in the wavelength range from 455 nm to 780 nm.

Item 7. A pharmaceutical product according to any one of Items 1 to 6, wherein the pharmaceutical preparation further comprises at least one member (B) selected from the group consisting of berberine, B2 vitamins, hesperidin, oxyquinoline, B12 vitamins, derivatives thereof, and salts thereof.

Item 8. A pharmaceutical product according to any one of Items 1 to 7, wherein the pharmaceutical preparation is an aqueous preparation.

Item 9. A pharmaceutical product according to Item 8, wherein tranilast and/or a salt thereof is present in a total proportion of 0.01 to 20% by weight based on the total amount of the pharmaceutical preparation.

Item 10. A pharmaceutical product according to any one of Items 1 to 9, wherein the pharmaceutical preparation is an eye drop, eye wash, injection, externally applied skin medicine, nasal drop, or contact lens-care formulation.

Item 11. A pharmaceutical product, comprising a packaging container (i) and a pharmaceutical preparation (ii), wherein

the packaging container (i) is a material through which the contents are visible and which blocks light in the wavelength range from 365 nm to 430 nm, and

the pharmaceutical preparation (ii) comprises tranilast and/or a salt thereof, and is contained in the packaging container (i).

The invention also provides the following methods for preventing photodegradation.

Item 12. A method for inhibiting photodegradation of tranilast or a salt thereof, comprising placing a pharmaceutical preparation comprising at least one member selected from the group consisting of tranilast and a salt thereof in a packaging container through which the contents are visible and which blocks light in the wavelength range from 365 nm to 430 nm.

Item 13. A method according to Item 12, wherein the packaging container has an average light transmittance of 20% or lower in the wavelength range from 365 nm to 430 nm.

Item 14. A method according to Item 13, wherein the packaging container has an average light transmittance of 20% or lower in the wavelength range from 350 nm to 430 nm.

Item 15. A method according to Item 13, wherein the packaging container has an average light transmittance of 20% or lower in the wavelength range from 350 nm to 450 nm.

Item 16. A method according to any one of Items 12 to 15, wherein the packaging container has an average light transmittance of 20% or lower in the wavelength range from 365 nm to 430 nm, 20% or lower in the wavelength range from 350 nm to 430 nm, and 20% or lower in the wavelength range from 350 nm to 430 nm.

Item 17. A method according to any one of Items 12 to 16, wherein a part of the packaging container has an average light transmittance of 30% or higher in the wavelength range from 455 nm to 780 nm.

Item 18. A method according to any one of Items 12 to 17, wherein the pharmaceutical preparation further comprises at least one member (B) selected from the group consisting of berberine, B2 vitamins, hesperidin, oxyquinoline, B12 vitamins, derivatives thereof, and salts thereof.

Item 19. A method according to any one of Items 12 to 18, wherein the pharmaceutical preparation is an aqueous preparation.

Item 20. A method according to Item 19, wherein tranilast and/or a salt thereof is present in a proportion of 0.01 to 20% by weight based on the total amount of the pharmaceutical preparation.

Item 21. A method according to any one of Items 12 to 20, wherein the pharmaceutical preparation is an eye drop, eye wash, injection, externally applied skin medicine, nasal drop, or contact lens-care formulation.

Item 22. A method for photostabilizing a pharmaceutical product comprising tranilast and/or a salt thereof, comprising putting a pharmaceutical preparation (ii) comprising tranilast and/or a salt thereof in a packaging container (i) through which the contents are visible and which blocks light in the wavelength range from 365 nm to 430 nm.

Further, according to the invention, the use of a transparent packaging container which blocks light in the wavelength range from 365 nm to 430 nm inhibits the degradation of at least one member selected from the group consisting of tranilast and a salt thereof when exposed to light. Thus, the prevention provides the following uses.

Item 23. Use of the packaging container through which the contents are visible and which blocks light in the wavelength range from 365 nm to 430 nm for inhibiting the degradation of at least one member selected from the group consisting of tranilast and a salt thereof when exposed to light.

Item 24. Use of the packaging container according to Item 23, wherein the packaging container has an average light transmittance of 20% or lower in the wavelength range from 360 nm to 430 nm.

Item 25. Use of the packaging container according to Item 24, wherein the packaging container has an average light transmittance of 20% or lower in the wavelength range from 350 nm to 430 nm.

Item 26. Use of the packaging container according to Item 24, wherein the packaging container has an average light transmittance of 20% or lower in the wavelength range from 350 nm to 450 nm.

Item 27. Use of the packaging container according to any one of Items 23 to 26, wherein the packaging container has an average light transmittance of 20% or lower in the wavelength range from 365 nm to 430 nm, 20% or lower in the wavelength range from 350 nm to 430 nm, and 20% or lower in the wavelength range from 350 nm to 430 nm.

Item 28 Use of the packaging container according to any one of Items 23 to 27, wherein a part of the packaging container has an average light transmittance of 30% or higher in the wavelength range from 455 nm to 780 nm.

Still further, the invention can prevent the degradation of a pharmaceutical preparation comprising at least one member selected from the group consisting of tranilast and a salt thereof when exposed to light and ensuring photostability with the use of a packaging container through which the contents are visible and which blocks light in the wavelength range from 365 nm to 430 nm. Thus, the prevention provides the following uses.

Item 29. Use of the packaging container through which the contents are visible and which blocks light in a wavelength range from 365 nm to 430 nm for preventing the degradation of a pharmaceutical preparation comprising at least one member selected from the group consisting of tranilast and a salt thereof when exposed to light.

Item 30. Use of the packaging container according to Item 29, wherein the packaging container has an average light transmittance of 20% or lower in the wavelength range from 360 nm to 430 nm.

Item 31. Use of the packaging container according to Item 30, wherein the packaging container has an average light transmittance of 20% or lower in the wavelength range from 350 nm to 430 nm.

Item 32. Use of the packaging container according to Item 30, wherein the packaging container has an average light transmittance of 20% or lower in the wavelength range from 350 nm to 450 nm.

Item 33. Use of the packaging container according to any one of Items 29 to 32, wherein the packaging container has an average light transmittance of 20% or lower in the wavelength range from 365 nm to 430 nm, 20% or lower in the wavelength range from 350 nm to 430 nm, and 20% or lower in the wavelength range from 350 nm to 430 nm.

Item 34. Use of the packaging container according to any one of Items 29 to 33, wherein a part of the packaging container has an average light transmittance of 30% or higher in the wavelength range from 455 nm to 780 nm.

### BRIEF DESCRIPTION OF THE DRAWINGS

FIG. 1 shows the light transmittance of a red film used in Test Example 1.

FIG. 2 shows the light transmittance of a yellow film used in Test Example 1.

FIG. 3 shows the light transmittance of a green film used in Test Example 1.

FIG. 4 shows the light transmittance of a yellow-green container made of polyethylene terephthalate used in Test Example 1.

FIG. 5 shows light transmittance of a reddish violet film used in Test Example 1.

FIG. 6 shows the light transmittance of a transparent glass container used in Test Example 1.

FIG. 7 shows the light transmittance of a transparent container made of polypropyrene used in Test Example 1.

FIG. 8 shows the light transmittance of a brown container made of polypropyrene used in Test Example 1.

FIG. 9 shows the light transmittance of a blue film used in Test Example 1.

FIG. 10 shows the light transmittance of a two-layered blue film used in Test Example 1.

FIG. 11 shows the light transmittance of a three-layered blue film used in Test Example 1.

### BEST MODE FOR CARRYING OUT THE INVENTION

In this specification, an aqueous pharmaceutical preparation refers to a pharmaceutical preparation comprising water in a proportion of 5% by weight or more, preferably 20% by weight or more, and further preferably 50% by weight or more based on the total amount of the pharmaceutical preparation.

I. Pharmaceutical Product

The present invention relates to a pharmaceutical product in which a pharmaceutical preparation containing tranilast and/or a salt thereof is held in a packaging container through which the contents are visible and which blocks light in the wavelength range from 365 nm to 430 nm.

More specifically, the invention relates to a pharmaceutical product, comprising a packaging container (i) and a pharmaceutical preparation (ii), in which the packaging container (i) is a material through which the contents are visible and which blocks light in the wavelength range from 365 nm to 430 nm, and the pharmaceutical preparation (ii) comprises tranilast and/or a salt thereof, and is held in the packaging container (i).

(i) Packaging Container

The packaging container used in the pharmaceutical product of the invention is a packaging container through which the contents are visible and which blocks light in the wavelength range from 365 nm to 430 nm.

In the invention, a “packaging container” refers to not only a packaging container directly holding a pharmaceutical preparation (hereafter referred to as “primary packaging container”) but one or more packaging containers (hereafter referred to as “secondary packaging container”) used to hold the primary packaging container directly holding the pharmaceutical preparation contained in.

When the pharmaceutical product of the invention has a secondary packaging container, all that is necessary is that at least the primary packaging container or the secondary packaging container of the pharmaceutical product blocks light in the above-mentioned wavelength ranges. From the viewpoint that the degradation of tranilast or a salt thereof held in a packaging container should be prevented not only during distribution and storage but during use, it is preferable that at least the primary packaging container blocks light in the above-mentioned wavelength ranges.

Any form of packaging container can be used without limitation insofar as the above-described pharmaceutical preparation can be contained therein, and the packaging container form can be suitably selected depending on the form and/or intended use of the pharmaceutical preparation to be held, or the intended use, i.e., a primary packaging container or a secondary packaging container, etc. Specific examples of forms of packaging containers for use as a primary packaging container include sachet-type (small-bag-type) packaging containers, tube-like containers, bottle-like containers, PTP packaging containers, eyedroppers, nasal droppers, etc. In addition, pillow packaging bags, etc., are mentioned as an example of forms of packaging containers for use as a secondary packaging container.

In the invention, a “packaging container through which the contents are visible” refers to a packaging container with content visibility, i.e., a packaging container having transparency to such a degree that the content held in the packaging container can be observed with the naked eye. Specific examples of a “packaging container through which the contents are visible” include packaging containers having an average light transmittance of 30% or higher, preferably 40% or higher, more preferably 50% or higher, and particularly preferably 70% or higher in a visible light range of 455 nm to 780 nm (hereinafter referred to as “average light transmittance in the 455 nm-780 nm wavelength range”). Packaging containers having the above-mentioned average light transmittance in the 455 nm-780 nm wavelength range are advantageous in that the visibility of the content in such packaging containers is ensured. In the invention, light transmittance is measured according to the method specified in JIS K7105. The average light transmittance in the 455 nm-780 nm wavelength range can be determined by measuring light transmittance every 5 nm in the wavelength range from 455 nm to 780 nm and computing an average value for the measured light transmittances.

In the invention, the pharmaceutical preparation in the packaging container is visible from the outside when the content visibility (transparency) of the packaging container is partially provided. Therefore, the content visibility (transparency) is not necessarily ensured over the entire surface of the packaging container. For example, in the case of a packaging container holding pharmaceutical preparations, such as eye drops and injections, the side thereof may have content visibility (transparency) of at least 80% or higher, and preferably 90% or higher from the viewpoint of determining the presence of insoluble foreign substances from the outside. The side of the container used herein refers to side portions, i.e., portions except a portion covered with a cap and the bottom in the entire surface of the container. Moreover, the content visibility (transparency) of the packaging container of the invention is not lessened even when a label for showing ingredients and a trade name is applied to the surface of the packaging container in the stage of, for example, distribution. However, it is preferable to apply the label in such a manner that the portion where the content visibility (transparency) is provided is sufficiently secured to allow a user to check the amount of pharmaceutical preparation contained in the packaging container.

In the invention, “blocking light in the wavelength range from 365 nm to 430 nm” refers to an average light transmittance in the wavelength range from 365 nm to 430 nm being 20% or lower (hereafter referred to as “average light transmittance in the 365 nm-430 nm wavelength range”). As in the case of the average light transmittance in the 455 nm-780 nm wavelength range, the average light transmittance in the 365 nm-430 nm wavelength range can be measured according to the method specified in JIS K7105. More specifically, it is determined by measuring the light transmittance every 5 nm in the wavelength range from 365 nm to 430 nm and computing an average value of the measured light transmittances.

From the viewpoint of more effectively inhibiting the photodegradation of tranilast and its salt, it is preferable to use a container whose average light transmittance in the wavelength range from 365 nm to 430 nm is preferably 15% or lower, more preferably 10% or lower, and particularly preferably 3% or lower.

In order to impart more excellent photostability to pharmaceutical preparations comprising tranilast and/or its salt, it is more preferable to use a container whose average light transmittance in the wavelength range from 350 nm to 450 nm (hereafter referred to as “average light transmittance in the 350 nm-450 nm wavelength range”) is 20% or lower, preferably 15% or lower, more preferably 10% or lower, still more preferably 5% or lower, and particularly preferably 3% or lower. The average light transmittance in the 350 nm-450 nm wavelength range is determined as in the case of the average light transmittance in the 365 nm-430 nm wavelength range.

Mentionable as one preferable aspect of the packaging container of the invention is, for example, a packaging container whose average light transmittance in the wavelength range from 350 nm to 450 nm (hereafter referred to as “average light transmittance in the 350 nm-450 nm wavelength range”) is 20% or lower, preferably 15% or lower, more preferably 10% or lower, still more preferably 5% or lower, and particularly preferably 3% or lower. The average light transmittance in the 350 nm-450 nm wavelength range is determined as in the case of the average light transmittance in the 365 nm-430 nm wavelength range.

In addition, mentionable as one preferable aspect of the packaging container of the invention is, for example, a packaging container in which the average light transmittance in each of the wavelength ranges from 365 nm to 430 nm, 350 nm to 450 nm, and 350 nm to 450 nm is 20% or lower, preferably 15% or lower, more preferably 10% or lower, and still more preferably 5% or lower.

Thus, with the packaging container capable of blocking light in the above-mentioned wavelength ranges to prevent the light from passing thereinto, the stability of tranilast or its salt in the packaging container can be ensured.

There is no limitation on the methods for blocking light in the wavelength ranges from 365 nm to 430 nm with the packaging container, insofar as the above-mentioned average light transmittance in the wavelength range from 455 nm to 780 nm is secured. For example, the light can be blocked by adding to a packaging container a substance capable of blocking light in the wavelength range from 365 nm to 430 nm.

There is no limitation on substances capable of blocking light in the wavelength range from 365 nm to 430 nm. Examples of such substances include Hansa pigments, benzimidazoline pigments, pyrazolone pigments, and like monozao pigments; diarylide pigments, pyrazorone pigments, and like disazo pigments; yellow pigments, and like condensation azo pigments; putalocyanine pigments, perinone pigments, isoindolinone pigments, anthraquinone pigments, and like condensation polycyclic pigments, etc. These substances capable of blocking light in the wavelength range from 365 nm to 430 nm may be used alone or in combination.

The following compounds (1) to (28) are mentioned as specific preferable examples of substances capable of blocking light in the wavelength range from 365 nm to 430 nm. Unless otherwise specified in this specification, compounds (1) to (26) are represented according to the Chemical Abstracts Service (CAS).
(1) benzenesulfonic acid, 4-[[3-[(dimethylphenyl)azo]-2,4-dihydroxyphenyl]azo]-;
(2) quinolinedisulfonic acid, 2-(1,3-dioxoindan-2-yl)-, (IUPAC: 2-(1,3-dioxoindan-2-yl)quinolinedisulfonic acid);
(3) quinoline, 2-(1,3-dioxoindan-2-yl), (IUPAC: 2-(1,3-dioxoindan-2-yl)-quinoline, (Generic name (color index name): Solvent Yellow 33);
(4) butanamide, 2,2′-[(3,3′-dichloro[1,1′-biphenyl]-4,4′diyl) bis(azo)]bis[3-oxo-N-phenyl-, (Generic name (color index name): Pigment Yellow 12);
(5) butanamide, 2-[(4-methyl-2-nitrophenyl)azo]-3-oxo-N-phenyl-, (Generic name (color index name): Pigment Yellow 1);
(6) benzenesulfonic acid, 5-chloro-2-[4,5-dihydro-3-methyl-4-[[4-[[(4-methylphenyl)sulfonyl]oxy]phenyl]azo]-5-oxo-1H-pyrazol-1-yl]-;
(7) 2-Naphthalenesulfonic acid, 8-hydroxy-5,7-dinitro-;
(8) 2-Naphthalenamine, 1-(phenylazo)-, (Generic name (color index name): Solvent Yellow 5);
(9) 2-Naphthalenamine, 1-(2-methylphenyl)azo]-, (Generic name (color index name): Solvent Yellow 6);
(10) Benzenesulfonic acid, 3-[[4-(phenylamino)phenyl]azo]-, (Generic name (color index name): Acid Yellow 36);
(11) Benzenesulfonic acid, 4-[4,5-dihydro-3-methyl-5-oxo-4-(phenylazo)-1H-pyrazol-1-yl]-, (Generic name (color index name): Acid Yellow 11);
(12) 3H-Pyrazol-3-one, 4,4′-[(3,3′-dichloro[1,1′-biphenyl]-4,4′-diyl)bis(azo)]bis[2,4-dihydro-5-methyl-2-phenyl-, (Generic name (color index name): Pigment Orange 13);
(13) Butanamide, 2-[(4-methoxy-2-nitrophenyl)azo]-N-(2-methylphenyl)-3-oxo-, (Generic name (color index name): Pigment Orange 1);
(14) Benzamide, 3,3′-[(2,5-dimethyl-1,4-phenylene)bis [imino(1-acetyl-2-oxo-2,1-ethanediyl)azo]]bis[4-chloro-N-(5-chloro-2-methylphenyl)-, (Generic name (color index name): Pigment Yellow 95;
(15) Benzamide, 3,3′-[(2,5-dimethyl-1,4-phenylene)bis[imino (1-acetyl-2-oxo-2,1-ethanediyl)azo]]bis[4-chloro-N-(2,5-dichlorophenyl)-, (Generic name (color index name): Pigment Yellow 166);
(16) Benzamide, 3,3′-[(2-chloro-5-methyl-1,4-phenylene)bis[imino (1-acetyl-2-oxo-2,1-ethanediyl)azo]]bis[4-chloro-N-(3-chloro-2-methylphenyl)-, (Generic name (color index name): Pigment Yellow 93);
(17) Benzenesulfonic acid, 4,5-dichloro-2-[[4,5-dihydro-3-methyl-5-oxo-1-(3-sulfophenyl)-1H-pyrazol-4-yl]azo]-;
(18) 1H-Isoindol-1-one, 3,3′-(1,4-phenylenediimino)bis [4,5,6,7-tetrachloro-, (Generic name (color index name): Pigment Yellow 110);
(19) 1H-Isoindol-1-one, 3,3′-[(2-methyl-1,3-phenylene)diimino]bis[4,5,6,7-tetrachloro-, (Generic name (color index name): Pigment Yellow 109);
(20) 1H-Isoindol-1-one, 4,5,6,7-tetrachloro-3-[[3-methyl-4-[[4-[(4,5,6,7-tetrachloro-1-oxo-1H-isoindol-3-yl)amino]phenyl]azo]phenyl]amino]-, (Generic name (color index name): Pigment Orange 61);
(21) Benzamide, 3,3′-[(2-chloro-5-methyl-1,4-phenylene)bis [imino(1-acetyl-2-oxo-2,1-ethanediyl)azo]]bis[4-chloro-N-[2-(4-chlorophenoxy)-5-(trifluoromethyl)phenyl]-, (Generic name (color index name): Pigment Yellow 128);
(22) 1,4-Benzenedicarboxylic acid, 2,2′-[1,4-phenylenebis[imino (1-acetyl-2-oxo-2,1-ethanediyl)azo]]bis-,tetramethyl ester, (Generic name (color index name): Pigment Yellow 155);
(23) 1H-Isoindol-1-one, 3,3′-[(2,5-dichloro-1,4-phenylene)diimino]bis-, (Generic name (color index name): Pigment Yellow 173);
(24) 9,10-Anthracenedione, 1,1′-[(6-phenyl-1,3,5-triazine-2,4-diyl)diimino]bis-, (Generic name (color index name): Pigment Yellow 147);
(25) Butanamide, 2,2′-[1,2-ethanediylbis(oxy-2,1-phenyleneazo)]bis [N-(2,3-dihydro-2-oxo-1H-benzimidazol-5-yl)-3-oxo-, (Generic name (color index name): Pigment Yellow 180);
(26) Benzamide, N-[4-(aminocarbonyl)phenyl]-4-[[1-[[(2,3-dihydro-2-oxo-1H-benzimidazol-5-yl)amino]carbonyl]-2-oxopropyl]azo]-, (Generic name (color index name): Pigment Yellow 181);
(27) Compounds represented by Formula (I); 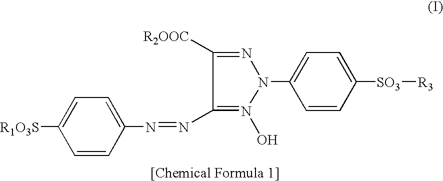
   wherein R₁, R₂, and R₃ are the same or different and each represents a hydrogen atom or an alkali metal atom. Examples of the alkali metal atom include a lithium atom, a sodium atom, a potassium atom, a rubidium atom, etc. Preferable among the compounds represented by Formula (I) are compounds represented by Formula (I) wherein R₁, R₂, and R₃ are the same or different and each represents a hydrogen atom, a sodium atom or a potassium atom. More preferable are compounds represented by Formula (I) wherein R₁, R₂, and R₃ are the same and each represents a sodium atom.
(28) Compounds represented by Formula (II): 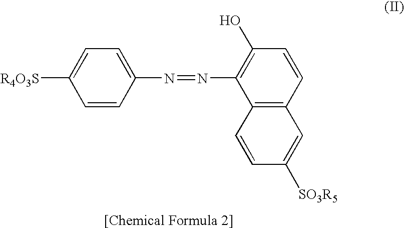
   wherein R₄ and R₅ are the same or different and each represents a hydrogen atom or an alkali metal atom. Examples of the alkali metal atom include a lithium atom, a sodium atom, a potassium atom, a rubidium atom, etc. Preferable among the compounds represented by Formula (II) are compounds represented by Formula (II) wherein R₄ and R₅ are the same or different and each represents a hydrogen atom, a sodium atom or a potassium atom. More preferable are compounds represented by Formula (II) wherein R₄ and R₅ are the same and each represents a sodium atom.

The above-described compounds of (1)-(26) can be used in the form of salts. Specific examples of the compounds in the form of salts include the following compounds:
(1-a) Benzenesulfonic acid, 4-[[3-[(dimethylphenyl)azo]-2,4-dihydroxyphenyl]azo]-, monosodium salt, (Generic name (color index name): Acid Orange 24);
(2-a) Quinoline disulfonic acid, 2-(1,3-dioxoindan-2-yl)-, sodium salt, (Generic name (color index name): Acid Yellow 3); (6-a) Benzenesulfonic acid,
5-chloro-2-[4,5-dihydro-3-methyl-4-[[4-[[(4-methylphenyl) suofonyl]oxy]phenyl]azo]-5-oxo-1H-pyrazole-1-yl]-, sodium salt, (Generic name (color index name):Acid Yellow 40);
(7-a) 2-naphthalene sulfonate, 8-hydroxy-5,7-dinitro-, disodium salt; Benzenesulfonic acid, 4-[4,5-dihydro-3-methyl-5-oxo-4-(phenylazo)-1H-pyrazole-1-yl]-, sodium salt, (Generic name (color index name): Acid Yellow 1); and
(17-a) Benzenesulfonic acid, 4,5-dichloro-2-[[4,5-dihydro-3-methyl-5-oxo-1-(3-sulfophenyl)-1H-pyrazole-4-yl]azo]-, calcium salt (1:1), (Generic name (color index name): Pigment Yellow 183).

Compounds (1) to (28) and salts thereof may be used in the form of a metal lake. Examples of metal for use in such a metal lake include barium, zirconia, calcium, magnesium, aluminum, etc.

As a substance blocking light in the wavelength range from 365 nm to 430 nm, compounds (1) to (28) and salts thereof can be used alone or in a combination thereof.

Compounds (1) to (28) are known, and moreover, such compounds can be prepared by known methods and are commercially available.

Specific examples of adding a substance that blocks light in the wavelength range from 365 nm to 430 nm to a packaging container include: a method comprising kneading, into a base material of a packaging container, a substance that blocks light in the wavelength range from 365 nm to 430 nm in a suitable amount, thereby preparing a packaging container comprising the substance; a method for applying, to an existing packaging container, a substance that blocks light in the wavelength range from 365 nm to 430 nm in a suitable amount; and a method for covering an existing packaging container with a film comprising a substance that blocks light in the wavelength range from 365 nm to 430 nm in a suitable amount. Examples of one preferable embodiment of a method for applying such a film to a container include a method comprising applying, to a packaging container, a thermal shrinkage film containing a substance that blocks light in the wavelength range from 365 nm to 430 nm in a suitable amount, and heating the resulting container to make the film adhere to the packaging container, providing a packaging container coated with the film.

When the packaging container for use in the invention is formed by kneading the substance that blocks light in the wavelength range from 365 nm to 430 nm into a packaging container material, the content of the substance(s) in the packaging container material is suitably determined according to the type of light-blocking substance and packaging container material, the form of the packaging container, etc. For example, in the case of a packaging container with a thickness of about 0.1 mm to about 1 mm, the proportion of light-blocking substance(s) is 0.001 to 15% by weight, preferably 0.001 to 10% by weight, and more preferably 0.01 to 5% by weight based on the total amount of packaging container material.

When a film kneaded with the substance that blocks light in the wavelength range from 365 nm to 430 nm is applied to a packaging container, the content of the light-blocking substance in the film varies depending on the type of the light-blocking substance, the type and thickness of the film, the form of the packaging container, etc. The total proportion of the light-blocking substance(s) is, for example, 0.0001 to 15% by weight, preferably 0.001 to 10% by weight, and more preferably 0.01 to 5% by weight.

Moreover, in the case of using compounds (1) to (28), it is preferable that the compound(s) is present in a portion with the surface area of 1 cm² of a packaging container where the content visibility (transparency) is provided and the total amount of the compound(s) is 0.001 mg to 20 mg, preferably 0.01 mg to 5 mg, and more preferably 0.1 mg to 1 mg.

The base material of the packaging container of the invention is not limited, and may be glass, plastic, cellulose, pulp, rubber, etc. When an aqueous preparation is contained, a plastic container is preferable as a packaging container in view of its squeezable property and durability.

Thermoplastic resin is preferable as a resin for the plastic packaging container for use in the invention, and examples of thermoplastic resin include olefin-based resin; polyester-based resin; polyphenylenether-based resin; polycarbonate-based resin; polysulfone-based resin; polyamide-based resin; rigid polyvinyl chloride resin; styrene-based resin, etc. When the packaging container is a plastic container, it is preferable to use a resin having a favorable squeezable property and the durability to withstand repeated pressing.

Moreover, the packaging container for use in the invention may further improve the photostability of tranilast or salts thereof, in combination with the effects of the invention, by adding a UV absorber, an infrared absorber, etc., to the resin or by applying a coating agent comprising such ingredient(s) to the surface of the resin.

(ii) Pharmaceutical Preparations

In a pharmaceutical product according to the invention, a pharmaceutical preparation is held in the above-described packaging container.

Tranilast contained in the pharmaceutical preparation refers to (N-(3,4-dimethoxycinnamoyl)anthranilic acid.

In place of or in combination with the above-mentioned tranilast, salts of tranilast can be used for the pharmaceutical preparation held in the packaging container. There is no limitation to tranilast salts insofar as the salts are pharmacologically acceptable. Specific examples thereof include inorganic salts such as sodium salt, potassium salt, or like; salts with organic amines such as morpholine, piperazine, piperidine, pyrrolidine, or like; salts with amino acids etc. These salts of tranilast may be used alone or in combination.

In the invention, there is no limitation to pharmaceutical preparations held in the packaging container insofar as tranilast and/or its salt are contained therein. For example, the pharmaceutical preparations may comprise only tranilast and/or its salt or may comprise, in addition to tranilast and/or its salt, pharmacologically acceptable bases, carriers, other pharmacological ingredients, other additives, etc.

In the invention, the content of tranilast and/or its salt in a pharmaceutical preparation held in the packaging container is suitably determined according to the intended use, dosage form, or the like of the pharmaceutical preparation. For example, when a pharmaceutical preparation is administered orally, daily dosage thereof is suitably determined in such a manner that the total amount of tranilast and its salt is adjusted within the range of 50 mg to 500 mg. In the case of a pharmaceutical preparation that is to be locally applied, the daily dosage thereof is suitably determined in such a manner that the total amount of tranilast and its salt is adjusted within the range of 0.01 mg to 50 mg. To be specific, in the case of an aqueous pharmaceutical preparation, the total proportion of tranilast and/or its salt is 0.01 to 20% by weight, preferably 0.01 to 10% by weight, and more preferably 0.1 to 5% by weight relative to the total amount of the pharmaceutical preparation.

The pharmaceutical preparation held in the packaging container may further comprise, in addition to (A) tranilast and/or its salt, (B)(B-1) berberine, derivatives thereof, or salts thereof (hereinafter referred to as “compounds (B-1)”); (B-2) B2 vitamins, derivatives thereof, or salts thereof (hereinafter referred to as “compounds (B-2)”); (B-3) hesperidin, derivatives thereof, or salts thereof (hereinafter referred to as “compounds (B-3)”); (B-4) oxyquinoline, derivatives thereof, or salts thereof (hereinafter referred to as “compounds (B-4)”); and (B-5) B12 vitamins, derivatives thereof, or salts thereof (hereinafter referred to as “compounds (B-5)”). Hereinafter, these compounds are referred to as “compounds (B)”. These compounds (B) may be used alone or in combination. The addition of one or more of these compounds (B) to the pharmaceutical product of the invention can further improve the photostability of tranilast and/or its salt contained in the pharmaceutical product of the invention.

Specific examples of compounds (B-1) include berberine, berberine tannate, berberine chloride, berberine sulfate, etc.

Specific examples of compounds (B-2) include flavin adenine dinucleotide, flavin mononucleotide, riboflavin, riboflavin phosphate, riboflavin acetate, riboflavin butyrate, sodium salt thereof, etc.

Specific examples of compounds (B-3) include hesperidin, methyl hesperidin, etc.

Specific examples of compounds (B-4) include oxyquinoline, oxyquinoline sulfate, oxyquinoline phosphate, etc.

Specific examples of compounds (B-5) include cyanocobalamin, mecobalamin, cobamamide, hydroxocobalamin, its hydrochloride, its acetate, etc.

The pharmaceutical preparation for use in the invention may contain at least one of compounds (1) to (28) mentioned in (i) above relating to the packaging container insofar as the compound is pharmacologically acceptable, thereby ensuring the photostabilization of tranilast or its salt also during formulation of pharmaceutical preparations.

The pharmaceutical preparation for use in the invention may comprise a solubilizer, if required. In particular, when a pharmaceutical preparation is formed into an aqueous pharmaceutical preparation, a solubilizer is preferably mixed therein. Mentioned as such solubilizers are trometamol, monoethanolamine, diethanolamine, triethanolamine, or like organic amines; polyvinylpyrrolidone; polysorbate or like surfactants; propylene glycol or like polyhydric alcohol; caffeine or like xanthine derivatives; or the like. Among the above, polyvinylpyrrolidone, caffeine, trometamol, monoethanolamine, diethanolamine, and triethanolamine have an action of enhancing the effect of the above-mentioned compound (B), i.e., improving the photostability of tranilast, and thus are preferable particularly when the above-mentioned compound(s) (B) is contained in the pharmaceutical preparation. These solubilizers can be used alone or in combination.

In the case of an aqueous pharmaceutical preparation, the content of the solubilizer, if mixed therein, is, for example, generally 0.001 to 10% by weight, preferably 0.05 to 10% by weight, and more preferably 0.1 to 5% by weight, based on the total amount of the pharmaceutical preparation.

By adding a specific nonionic surfactant into the pharmaceutical preparation for use in the invention, the photostability of tranilast and its salt can be more effectively improved. Suitable examples of nonionic surfactants include POE-POP block copolymer, POE sorbitan fatty acid ester, POE castor oil, or POE hardened castor oil. In particular, POE (60) hardened castor oil, polysorbate 80, and poloxamer 407 are preferable. Herein, the number in parentheses represents the number of added moles (average number of added moles), and “POE” and “POP” are abbreviations for polyoxyethylene and polyoxypropylene, respectively. These nonionic surfactants can be used alone or in combination.

In the case of an aqueous pharmaceutical preparation, the content of the nonionic surfactant therein is generally about 0.001 to 1% by weight, preferably about 0.01 to 1% by weight, and more preferably about 0.05 to 1% by weight based on the total amount of the pharmaceutical preparation.

When a chelating agent is further mixed into the pharmaceutical preparation for use in the invention, more excellent photostability can be imparted to tranilast and its salt contained in the pharmaceutical preparations. Suitable examples of such a chelating agent include ethylenediaminetetraacetic acid, citric acid, and salts thereof. Particularly preferable as chelating agents are ethylenediaminetetraacetic acid and salts thereof. Specific examples thereof include ethylenediaminetetraacetic acid disodium, ethylenediaminetetraacetic acid disodium dihydrate (edetate sodium), etc. These chelating agents can be used alone or in combination.

The chelating agent content is, for example, 0.0001 to 10 parts by weight, preferably 0.001 to 10 parts by weight, more preferably 0.001 to 1 part by weight, and particularly preferably 0.01 to 1 part by weight, when the total amount of tranilast and salts thereof is 1 part by weight.

When the pharmaceutical preparation for use in the invention is an aqueous pharmaceutical preparation, a specific buffer is mixed into the pharmaceutical preparation in place of or in combination with the chelating agent, thereby imparting more excellent photostability to tranilast and salts thereof contained in the pharmaceutical preparation. Examples of such buffers include borate buffer, phosphate buffer, carbonate buffer, etc. Among the above, borate buffer and phosphate buffer are preferable. The proportion of the buffer, if it is mixed in, in a pharmaceutical preparation is generally 0.0001 to 10% by weight, preferably 0.001 to 5% by weight, more preferably 0.01 to 5% by weight, and particularly preferably about 0.1 to about 3% by weight, in terms of the concentration of the total amount of the buffer.

When the pharmaceutical preparation for use in the invention is an aqueous pharmaceutical preparation, the pH thereof is usually adjusted within the range of 4 to 9. To maintain favorable photostability of tranilast and/or salts thereof, the pH is preferably 6 to 8.5, more preferably 7 to 8.5, and particularly preferably 7 to 8. In the case of an aqueous pharmaceutical preparation, the osmolar ratio, which is the ratio of the osmolarity of the pharmaceutical preparation to that of physiological saline is generally within the range of 0.3 to 4.1, preferably 0.3 to 2.1, and particularly preferably about 0.5 to about 1.4.

The pharmaceutical preparation for use in the invention is adjusted into various dosage forms according to the intended use in combination with various carriers, such as aqueous carriers, hydrophilic carriers, oil carriers, liquid carriers, powder/granule carriers, etc. The following various forms are mentioned as dosage forms of the pharmaceutical preparation of the invention: tablets, powders, granules, capsules, dry syrups, or like solid dosage forms; hard ointments, soft ointments, creams, or like semi-solid dosage forms; and eye drops, nasal drops, lotions, extracts, suspensions, emulsions, syrups, injections (including injections prepared before use), aerosols, soft capsules, drinks, or like liquid dosage forms; etc. Aqueous dosage forms can be mentioned as a particularly preferable dosage form of the pharmaceutical preparation of the invention in view of the fact that the invention effectively prevents the destabilization of tranilast and/or its salt when exposed to light. Examples of such aqueous pharmaceutical preparations include liquid agents and semi-solid agents containing water.

The pharmaceutical preparation for use in the invention may be injections, suppositories, oral medicines, preparations for inhalation, etc., or may be topically applied to a part of the body that is subjected to light exposure during application. The pharmaceutical preparation of the invention may be applied not only to the skin but to membranes that are sensitive to stimulation (ocular-mucous membranes, such as cornea, conjunctiva, etc.; gums; tongue; lips; oral mucosa; nasal mucous membrane; pharyngeal mucosa; etc.). Specific examples of the pharmaceutical preparation for use in the invention include externally applied ointments, externally applied creams, externally applied liquids, or like externally applied skin-care preparations; eye drops; eye washes; ophthalmic ointments; contact lens-wearing solutions; contact lens-care formulations (such as cleaning solutions, soaking solutions, disinfecting solutions, multi-purpose solutions, etc.); nasal drops; nasal lavage fluids; oropharyngeal therapeutic drugs; gargles; ear drops; etc. Among the above, eye drops, eye washes, injections, externally applied skin-care preparations, nasal drops, and contact lens-care formulations are preferable.

The invention makes it possible to visually observe the pharmaceutical preparation contained in the packaging container from the outside thereof with the naked eye so as to check the presence of a foreign substance. Therefore, the invention is advantageous in facilitating the quality control of pharmaceutical preparations, in particular, such as eye drops, eye washes, and injections. Moreover, the invention is also advantageous in that a user can check the amount of pharmaceutical preparation remaining in the packaging container from the outside thereof with the naked eye during each use when two or more doses of the pharmaceutical preparation are charged in the packaging container. Examples of pharmaceutical preparations to be charged in the packaging container in two or more doses include eye drops, eye washes, externally applied skin-care preparations, nasal drops, and contact lens-care formulations.

The pharmaceutical preparations for use in the invention may comprise pharmacologically active ingredients and/or bioactive ingredients together with the above-mentioned ingredient(s) insofar as the effect of the invention is not adversely affected.

As regards the pharmaceutical product of the invention, the amount of the pharmaceutical preparation to be contained in the packaging container can be suitably determined according to a single dose of the pharmaceutical preparation, the number of doses of the pharmaceutical preparation per packaging container, etc.

II. Method for Inhibiting Photodegradation

As described above, the packaging container which allows a user to visually observe the content and which blocks light in the wavelength range from 365 nm to 430 nm is able to inhibit the photodegradation of tranilast and salts thereof. Taking a different perspective from the above description, the invention provides a method for inhibiting the photodegradation of tranilast and salts thereof, the method comprises housing (i) a pharmaceutical preparation containing tranilast and/or salts thereof in (ii) a packaging container that blocks light in the wavelength range from 365 nm to 430 nm.

In the method of the invention, the type of tranilast and salts thereof to be used, the content thereof in a pharmaceutical preparation, other ingredients mixed in a pharmaceutical preparation, the form of a pharmaceutical preparation, a packaging container blocking light in the wavelength range from 365 nm to 430 nm apply in the same way as in the above-described pharmaceutical preparation contained in the packaging container.

### EXAMPLES

Hereinafter, the invention will be described in more detail with reference to Examples, but is not limited thereto.

### Test Example 1

### Confirmatory Test for Tranilast-Degradation Inhibition

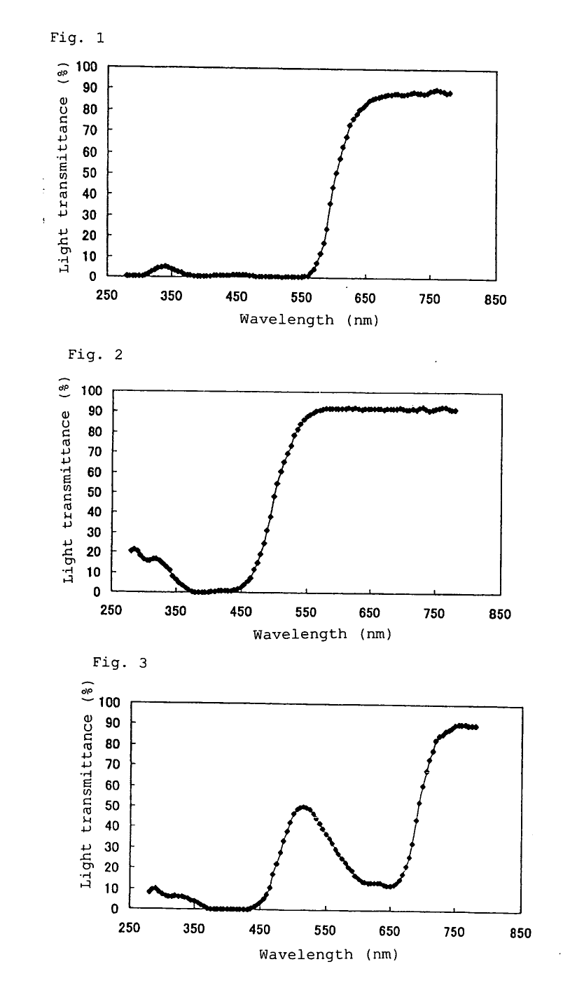

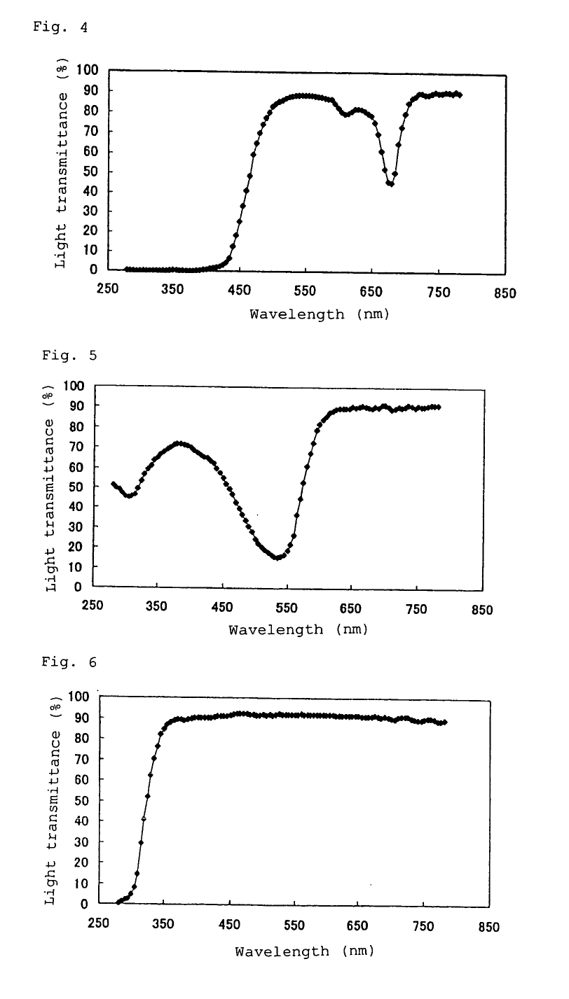

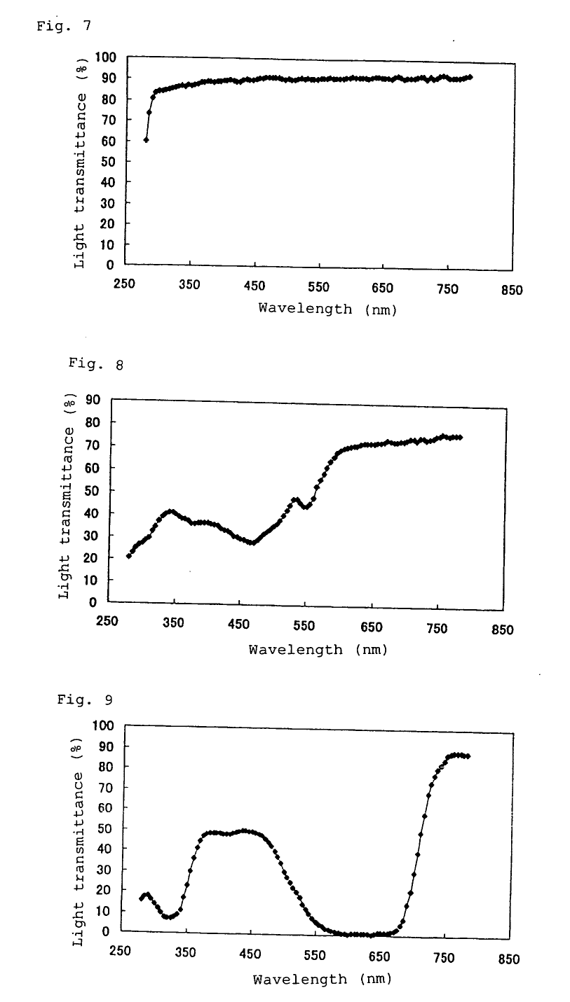

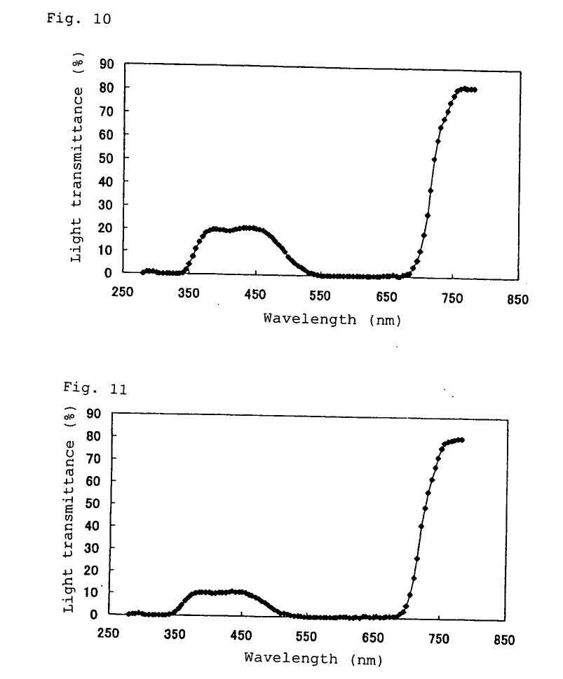

A tranilast-containing pharmaceutical preparation having the formulation shown in Table 1 (Formulation 1) was prepared, and then filtered through a 0.2 μm membrane filter. Subsequently, the tranilast-containing pharmaceutical preparation was dispensed in 10 ml portions into several 10 ml containers made of various materials shown in Table 2, and the containers were then sealed. Each tranilast-containing pharmaceutical preparation held in the containers thus obtained was continuously irradiated with light of 5000 lux at 25° C. (room temperature) for 60 hours with a photostability testing device comprising a D65 fluorescent lamp as a light source (trade name “Light-Tron LT-120 D3CJ type”, product of Nagano Science Co., Ltd.), and thus the accumulated light exposure of preparation was 300000 lux·hr. Thereafter, the tranilast concentration of each tranilast-containing pharmaceutical preparation was analyzed by high-performance liquid chromatography to thereby determine the residual concentration of tranilast. The residual ratio (%) of tranilast relative to the concentration of tranilast before light irradiation was computed based on the measured residual concentration of tranilast. In order to evaluate the light transmittance of the containers and the various films applied to the containers used in this test, the containers and films were cut into planar shapes, providing samples. The samples were evaluated by Spectral Transmittance Meter for generally lens TM-1 (product of TOPCON) to determine the light transmittance in the wavelength range from 280 nm to 800 nm (see FIGS. 1 to 11). Thus, the average light transmittance of the wavelength ranges from 365 nm to 430 nm, 350 nm to 430 nm, and 350 nm to 450 nm were determined.

This test was performed using glass, polypropylene (PP), and polyethylene terephthalate (PET) containers. As light-blocking means, the test was performed using containers covered with various films and containers blended with brown or yellow-green pigment for coloring (see Table 2). Shrink films (heat shrinkable films) were applied to the entire exterior of the container in a routine manner, and other types of films were wrapped around the entire exterior of the container and then fixed with tape. The films used for this test were obtained as follows: red film (TOYO Corporation, red cellophane); yellow film (TOYO corporation, yellow cellophane); green film (TOYO Corporation, green cellophane); blue film (TOYO Corporation, blue cellophane), and reddish violet film (reddish violet cellophane).

The containers used for the test were evaluated for the content visibility according to following evaluation criteria.

<Evaluation Criteria>

⊚ The amount of inside contents and the presence of foreign substances were clearly visually observable.

◯ The amount of inside contents and the presence of foreign substances were visually observable.

Δ The amount of inside contents was visually observable but the presence of foreign substances was not be visually observable.

x The amount of inside contents was not visually observable.

TABLE 1

| Ingredient (g/100 mL) | Formulation 1 |
|---|---|
| Tranilast | 0.5 |
| Polyvinylpyrrolidone | 3.0 |
| Boric acid | 1.3 |
| Borax | 0.7 |
| Polysorbate 80 | 0.1 |
| Hydrochloric acid | suitable amount |
| Sodium hydroxide | suitable amount |
| Benzalkonium chloride | 0.005 |
| Purified water | suitable amount |
| Total amount | 100 ml |
| pH | 7.5 |
| Osmolar ratio | 1 |

The osmolar ratio is calculated by setting the osmotic pressure of physiological saline as defined in the Japanese Pharmacopoeia to 1.

Among the containers used in this test, the containers having 30% or higher average light transmittance in the wavelength range from 455 nm to 780 nm were sufficiently transparent so that the pharmaceutical preparation contained therein was visually observable from the outside, and thus are preferable as the container of the invention. Moreover, among the containers used in this test, the containers covered with a red, yellow, or green film and the yellow-green PET container exhibited 30% or higher average light transmittance in the wavelength range from 455 nm to 780 nm, and they effectively blocked light in the wavelength range from 365 nm to 430 nm (see FIGS. 1 to 4 and Table 2).

Table 2 also shows the measurement result of the residual ratio of tranilast after light irradiation. As can be seen from Table 2, every tranilast-containing pharmaceutical preparation held in a container blocking light in the wavelength range from 365 nm to 430 nm showed a notably high residual concentration of tranilast after light irradiation. It was also confirmed that tranilast is stably held in the containers whose average light transmittance is low not only in the wavelength range from 365 nm to 430 nm but also in the wavelength ranges from 350 nm to 450 nm and from 350 nm to 430 nm. In contrast, it was confirmed that degradation of tranilast caused by light irradiation is not inhibited in the tranilast-containing pharmaceutical preparations held in those containers having a high average light transmittance in the wavelength range from 365 nm to 430 nm.

The above results show that light in the wavelength range from 365 nm to 430 nm causes degradation of tranilast when exposed to light, and therefore, by blocking the light in this wavelength range, degradation of tranilast when exposed to light is inhibited. Moreover, it was also found that a favorable content visibility is ensured in a tranilast-containing pharmaceutical preparation held in a container having 30% or higher of average light transmittance in the wavelength range from 455 nm to 780 nm.

TABLE 2

| | | | | | | | | | Stability |
|---|---|---|---|---|---|---|---|---|---|
| | | Containers | | | | | | | Residual |
| | | | | Average light | Average light | Average light | Average light | | ratio of |
| | | | | transmittance of | transmittance | transmittance | transmittance | | tranilast |
| | | | | light in the | of light in the | of light in the | of light in the | | after |
| | | | | wavelength | wavelength | wavelength | wavelength | Visibility | irradiation |
| | | | | range from 365 | range from | range from | range from | Evaluation | of light of |
| | | Materials | Applied | nm to 430 nm | 350 nm to | 350 nm to 450 | 455 nm to | of content | 300000 lx |
| | | (coloring) | films | (%) | 430 nm (%) | nm (%) | 780 nm (%) | visibility | (%) |
| Examples | 1 | glass | red film | 1 | 1 | 1 | 48 | ◯ | 96.2 |
| | 2 | glass | yellow film | 1 | 1 | 1 | 78 | ⊚ | 93.4 |
| | 3 | PP | yellow film | 1 | 1 | 1 | 78 | ⊚ | 94.1 |
| | 4 | PP | yellow | 3 | 2 | 3 | 75 | ⊚ | 93.5 |
| | | | shrink film | | | | | | |
| | 5 | glass | green film | 0 | 1 | 1 | 42 | ◯ | 95.2 |
| | 6 | PET | — | 1 | 1 | 4 | 79 | ⊚ | 96.1 |
| | | (yellow | | | | | | | |
| | | green) | | | | | | | |
| Comparative | 1 | glass | — | 90 | 90 | 90 | 92 | ⊚ | 9.8 |
| Examples | 2 | PP | — | 89 | 90 | 89 | 91 | ⊚ | 8.2 |
| | 3 | glass | reddish | 69 | 68 | 67 | 66 | ⊚ | 23.8 |
| | | | violet film | | | | | | |
| | 4 | glass | blue film | 48 | 44 | 45 | 30 | ◯ | 42.8 |
| | 5 | glass | blue films | Not | 18 | 18 | 19 | Δ | 77.9 |
| | | | (2 pieces) | determined | | | | | |
| | 6 | glass | blue films | 10 | 9 | 9 | 16 | Δ | 93 |
| | | | (3 pieces) | | | | | | |
| | 7 | PP | — | 36 | 36 | 35 | 56 | ◯ | 33.3 |
| | | (brown) | | | | | | | |

In Table 2, the mark “-” in the column of “applied films” means no applied film. In the column of “applied films”, “blue films (2 pieces)” means that two blue films were applied to the container and “blue films (3 pieces)” means that three blue films were applied to the container.

### Test Example 2

A tranilast-containing pharmaceutical preparation having the formulation shown in Table 1 (Formulation 1) was prepared, and then filtered through a 0.2 μm membrane filter.

Separately prepared were an aqueous solution (test sample A) comprising 0.01% by weight of the compound represented by Formula (I) wherein R₁, R₂, and R₃ each represent a sodium atom; an aqueous solution (test sample B) comprising 0.1% by weight of the compound represented by Formula (I) wherein R₁, R₂, and R₃ each represent a sodium atom; an aqueous solution (test sample C) comprising 0.01% by weight of the compound represented by Formula (II) wherein R₄ and R₅ each represent a sodium atom; and an aqueous solution (test sample D) comprising 0.1% by weight of the compound represented by Formula (II) wherein R₄ and R₅ each represent a sodium atom.

The tranilast-containing pharmaceutical preparations prepared above were dispensed in 10 mL portions into several 10 mL screw cap bottles (outer diameter of 25 mm; trade name “SV-10”; product of NICHIDEN-RIKA GLASS CO., LTD.), and the bottles were sealed, preparing several airtight containers holding a tranilast-containing pharmaceutical preparation. Subsequently, each airtight container holding a tranilast-containing pharmaceutical preparation was placed in a 50 mL screw cap bottle (inner diameter of 35 mm; trade name “SV -50”; product of NICHIDEN-RIKA GLASS CO., LTD.). One of the test samples A to D prepared above was added to each of the 50 mL screw cap bottles up to the maximum capacity in such a manner as to surround the airtight container holding the tranilast-containing pharmaceutical preparation (Examples 7-10). Using the pharmaceutical preparation-containing 50 mL screw cap bottles thus prepared as a model of a tranilast-containing pharmaceutical preparation held in a packaging container, the following tests were performed. With each tranilast-containing pharmaceutical preparation held in a packaging container used as a test model, the amount of tranilast-containing pharmaceutical preparation and the presence of foreign substances were visually observable from outside the packaging container.

Each pharmaceutical preparation-containing 50 mL screw cap bottle above (Examples 7 to 10) was continuously irradiated with light of 5000 lux at 25° C. (room temperature) for 60 hours with a photostability testing device comprising a D65 fluorescent lamp as a light source (trade name “Light-Tron LT-120 D3CJ type”, product of Nagano Science Co., Ltd.), and thus the accumulated light exposure of each tranilast-containing pharmaceutical preparation was 300000 lux·hr. Thereafter, each tranilast-containing pharmaceutical preparation was subjected to high-performance liquid chromatography to thereby determine the residual concentration of tranilast. The residual ratio (%) of tranilast relative to the concentration of tranilast before light irradiation was computed based on the measured residual concentration of tranilast.

For comparison, a test sample comprising water (comparative example 8) was similarly tested.

Table 3 shows the results. The results show that when the tranilast-containing pharmaceutical preparation is covered by a compound represented Formula (I) or (II), the optical decomposition of tranilast is sharply inhibited.

TABLE 3

| | | Residual ratio |
|---|---|---|
| | Test sample used | of tranilast |
| Ex. 7 | Test sample A: | 90.6% |
| | [an aqueous solution comprising 0.01% by weight of the compound represented | |
| | by Formula (I) wherein R₁, R₂, and R₃ each represent a sodium atom] | |
| Ex. 8 | Test sample B: | 98.2% |
| | [an aqueous solution comprising 0.1% by weight of the compound represented | |
| | by Formula (I) wherein R₁, R₂, and R₃ each represent a sodium atom] | |
| Ex. 9 | Test sample C: | 81.3% |
| | [an aqueous solution comprising 0.01% by weight of the compound represented | |
| | by Formula (II) wherein R₄ and R₅ each represent a sodium atom] | |
| Ex. 10 | Test sample D: | 97.8% |
| | [an aqueous solution comprising 0.1% by weight of the compound represented | |
| | by Formula (II) (wherein R₄ and R₅ each represent a sodium atom)] | |
| Com. Ex. 8 | Water | 10.2% |

### INDUSTRIAL APPLICABILITY

The invention provides a pharmaceutical product in which a pharmaceutical preparation comprising tranilast and/or a salt thereof is held in a packaging container whose content visibility is ensured. In the pharmaceutical product, the pharmaceutical preparation contained in the packaging container can be observed with the naked eye, and further, degradation of tranilast due to light exposure is sharply inhibited. Thus, according to the invention, the production process control and quality control of the pharmaceutical preparation comprising tranilast and/or a salt thereof can be made reliable, and the storage stability of the tranilast-containing pharmaceutical preparation can be improved.

According to the method for inhibiting the photodegradation of tranilast and a salt thereof of the invention, the photodegradation of tranilast and/or a salt thereof can be sharply inhibited by merely placing a pharmaceutical preparation comprising tranilast and/or a salt thereof in a packaging container which blocks light in the specific wavelength range and whose content visibility is ensured. Thus, according to the method of the invention, photodegradation of tranilast and a salt thereof can be inhibited and moreover the pharmaceutical preparation in the packaging container can be evaluated with the naked eye for its amount, state and also the presence of insoluble foreign substance. Thus, the invention can facilitate quality control during production of a tranilast-containing pharmaceutical preparation. Moreover, the pharmaceutical preparation held in the packaging container obtained by the method of the invention is advantageous in that a user can, before use, check with the naked eye the amount and state of the contents from outside the packaging container.

1. A pharmaceutical product, comprising a pharmaceutical preparation containing at least one member selected from the group consisting of tranilast and a salt thereof in a packaging container through which the contents are visible and which blocks light in the wavelength range from 365 nm to 430 nm.

2. A pharmaceutical product according to claim 1, wherein the packaging container has an average light transmittance of 20% or lower in the wavelength range from 365 nm to 430 mm.

3. A pharmaceutical product according to claim 2, wherein the packaging container has an average light transmittance of 20% or lower in the wavelength range from 350 nm to 430 nm.

4. A pharmaceutical product according to claim 2, wherein the packaging container has an average light transmittance of 20% or lower in the wavelength range from 350 nm to 450 nm.

5. A pharmaceutical product according to claim 1, wherein the packaging container has an average light transmittance of 20% or lower in the wavelength range from 365 nm to 430 nm, 20% or lower in the wavelength range from 350 nm to 430 nm, and 20% or lower in the wavelength range from 350 nm to 450 nm.

6. A pharmaceutical product according to claim 1, wherein the packaging container has an average light transmittance of 30% or higher in the wavelength range from 455 nm to 780 nm.

7. A pharmaceutical product according to claim 1, wherein the pharmaceutical preparation further comprises at least one member selected from the group consisting of berberine, B2 vitamins, hesperidin, oxyquinoline, B12 vitamins, derivatives thereof, and salts thereof.

8. A pharmaceutical product according to claim 1, wherein the pharmaceutical preparation is an aqueous preparation.

9. A pharmaceutical product according to claim 8, wherein tranilast and a salt thereof is present in a total proportion of 0.01 to 20% by weight based on the total amount of the pharmaceutical preparation.

10. A pharmaceutical product according to claim 1, wherein the pharmaceutical preparation is an eye drop, eye wash, injection, externally applied skin medicine, nasal drop, or contact lens-care formulation.

11. A method for inhibiting photodegradation of tranilast or a salt thereof, comprising placing a pharmaceutical preparation comprising at least one member selected from the group consisting of tranilast and a salt thereof in a packaging container through which the contents are visible and which blocks light in the wavelength range from 365 nm to 430 nm.

12. Use of the packaging container through which the contents are visible and which blocks light in the wavelength range from 365 nm to 430 nm for inhibiting the degradation of at least one member selected from the group consisting of tranilast and a salt thereof when exposed to light.

13. Use of the packaging container through which the contents are visible and which blocks light in a wavelength range from 365 nm to 430 nm for preventing the degradation of a pharmaceutical preparation comprising at least one member selected from the group consisting of tranilast and a salt thereof when exposed to light.
